(19) United States
(12) Patent Application Publication
(10) Pub. No.: US 2003/0158090 A1
(43) Pub. Date: Aug. 21, 2003

(54) Renin-angiotensin system in diabetes mellitus

(76) Inventors: Ulrik Pedersen-Bjergaard, Hillerod (DK); Birgit Agerholm-Larsen, Birkerod (DK); Birger Thorsteinsson, Hellerup (DK); Stig Pramming, Copenhagen K (DK)

Correspondence Address: JACOBSON HOLMAN PLLC, 400 SEVENTH STREET N.W., SUITE 600, WASHINGTON, DC, 20004, US

(21) Appl. No.: 10/195,330

(22) Filed: Oct. 4, 2002

Related U.S. Application Data
(60) Provisional application No. 60/306,859, filed on Jul. 23, 2001.

(51) Int. Cl. A61K 38/28; A61K 38/05; A61K 31/4184; A61K 31/4178

(52) U.S. Cl. 514/3; 514/19; 514/381

(57) ABSTRACT

The present invention provides novel methods of treatment of diabetes mellitus as well as methods of diagnosing the susceptibility of hypoglycaemia in an individual. The method of treatment includes administering to an individual a sufficient amount of at least one inhibitor of the renin-angiotensin II system and at least one antidiabetic, for example insulin. Another objective of the present invention is to provide methods of preventing hypoglycaemia in an individual in need thereof comprising administering to said individual a pharmaceutical effective amount of an inhibitor of the renin-angiotensin II system. In particular, such an individual may be an individual suffering from diabetes mellitus. A further objective of the present invention is to provide methods to diagnose the susceptibility to hypoglycaemia of an individual comprising detecting within a predetermined tissue sample the genotype of the angiotensin-converting enzyme (ACE) gene; or detecting within a predetermined tissue sample the activity of ACE; and correlating said genotype or activity to the susceptibility of hypoglycaemia.

This application is a non-provisional of U.S. provisional application Serial No. 60/306,859 filed on Jul. 23, 2001, which is hereby incorporated by reference in its entirety.

All patent and non-patent references cited in the application, or in the present application, are also hereby incorporated by reference in their entirety.

### TECHNICAL FIELD

The present invention provides novel pharmaceutical compositions for the treatment of diabetes mellitus. Furthermore, the invention provides novel methods of treatment of diabetes mellitus as well as methods of diagnosing the susceptibility of hypoglycaemia in an individual.

### BACKGROUND OF THE INVENTION

Diabetes mellitus is a syndrome characterised by insufficient insulin secretion and reduced glucose tolerance. Moreover, individuals suffering from diabetes mellitus have a significant increased risk of contacting arteriosclerosis. The two major forms of the disease are type 1 and type 2 diabetes. Type 2 diabetes, the most common form, usually appears in adults, often in middle age. Type 2 diabetes is often associated with obesity and may be delayed or controlled with diet and exercise. Obesity and physical inactivity are two risk factors for type 2 diabetes. The other form of diabetes mellitus is type 1 or juvenile diabetes. It typically begins early in life. Individuals suffering from type 1 diabetes have a primary insulin deficiency and are dependent on insulin treatment throughout life.

Hypoglycaemia i.e. low blood sugar is a complication, which can be caused by diabetes treatment. Severe hypoglycaemia, i.e. hypoglycaemic episodes where the patient is dependent on assistance from others, is a major limitation for the achievement of optimal glycaemic control in patients with type 1 diabetes mellitus (1,2).

Since modern diabetes treatment aims at keeping blood glucose levels near normal to reduce the risk of late complications (3,4), prediction of severe hypoglycaemia has become increasingly important. However currently, no efficient method to predict an individuals susceptibility to hypoglycaemia is available.

Blunted secretion of counterregulatory hormones and loss of warning symptoms of hypoglycaemia are the two strongest known predictors of severe hypoglycaemia (2,5). It is, however, a well-known clinical observation that a number of type 1 diabetic patients with impaired counterregulation or loss of hypoglycaemic warning symptoms do not suffer from severe hypoglycaemia. Thus, it seems as if susceptibility to severe hypoglycaemia is highly variable between patients. This variability is a main obstacle of predicting severe hypoglycaemia in patients with type 1 diabetes.

Furthermore, no treatment is currently available to decrease the risk of severe hypoglycaemia in a diabetic patient. As outlined above it would be of great value to provide a method which could reduce the risk of hypoglycaemia in diabetic patients.

The renin-angiotensin system comprise a number of components which are being produced mainly in the kidney cortex and which have a vasoconstricting effect both locally as well as generally on the peripheral arterioles. Angiotensinogen is a plasmaprotein synthesised in the liver. Angiotensinogen is converted to the decapeptide angiotensin I by the enzyme renin, which is produced by the epithelial cells of the afferent arterioles. Angiotensin I is further processed to angiotensin II by the angiotensin converting enzyme (ACE). Angiotensin II is an octapeptide with several physiological effects i.e. angiotensin II is a potent vasoconstrictor. The sequence of angiotensin II is:

Asp-Arg-Val-Tyr-Ile-His-Pro-Phe

Several pharmacological inhibitors of the renin-angiotension system are known. In particular such inhibitors are inhibitors of the angiotensin converting enzyme (ACE) or they are antagonists of the angiotensin II receptor.

Recently, it has been indicated that a polymorphism of the angiotensin converting enzyme (ACE) gene may be associated with performance of endurance athletes (6). The polymorphism consists of an I (insertion) and a D (deletion) allele, the former conferring low tissue and blood activity of ACE (7,8), probably due to the I allele's production of an ACE protein that has only one of its two putative active sites (9). An increased frequency of the I allele has been demonstrated in high-altitude mountaineers (6), in Australian Olympic rowers (10), and in British Olympic long-distance runners (11).

### SUMMARY OF THE INVENTION

The present invention demonstrates that individuals with high ACE activity suffering from diabetes have an increased susceptibility to hypoglycaemia and surprisingly it is disclosed that it is possible to use inhibitors of the renin-angiotensin system in treatment of diabetes to decrease the risk of hypoglycaemia.

Accordingly, it is a first objective of the present invention to provide a kit-of-parts for the treatment of diabetes mellitus comprising

a) at least one inhibitor of the renin-angiotensin II system; and

b) at least one antidiabetic

It is a second objective of the present invention to provide methods of treatment of diabetes mellitus in an individual in need thereof comprising administering to said individual a pharmaceutical effective amount of a kit-of-parts comprising at least one inhibitor of the renin-angiotensin II system and at least one antidiabetic.

It is a third objective of the present invention to provide methods of preventing hypoglycaemia in an individual in need thereof comprising administering to said individual a pharmaceutical effective amount of an inhibitor of the renin-angiotensin II system. In particular, such an individual may be an individual suffering from diabetes mellitus.

It is a further objective of the present invention to provide methods to diagnose the susceptibility to hypoglycaemia of an individual comprising the steps of

a) Obtaining a tissue sample from the individual; and

b) Detecting within said tissue sample the genotype of the angiotensin-converting enzyme (ACE) gene; and

c) Correlating the genotype with the susceptibility to hypoglycaemia.

It is a still further objective of the present invention to provide methods to diagnose the susceptibility to hypoglycaemia of an individual comprising the steps of

a) Obtaining a tissue sample from the individual; and

b) Detecting within said tissue sample the activity of ACE; and

c) Correlating said activity to the susceptibility of hypoglycaemia.

It is yet another objective of the present invention to provide methods of treatment of diabetes mellitus in an individual in need thereof comprising the steps of

a) Diagnosing the susceptibility of said individual to hypoglycaemia by determining the ACE genotype of the individual and/or determining the activity of ACE in a tissue sample from the individual

b) Determining an appropriate dose of an antidiabetic and/or an inhibitor of the renin-angiotensin II system to treat said individual according to said diagnosis; and

c) Administering to said individual said appropriate dose of an antidiabetic

Furthermore, it is an objective of the present invention to provide uses of a pharmaceutical effective amount of the kit-of-parts comprising at least one inhibitor of the renin-angiotensin II system and at least one antidiabetic for the preparation of a medicament for the treatment of diabetes mellitus in an individual in need thereof.

It is furthermore an objective of the present invention to provide uses of a pharmaceutical effective amount of an inhibitor of the renin-angiotensin II system for the preparation of a medicament for the preventing hypoglycaemia in an individual in need thereof. In particular, such an individual may be an individual suffering from diabetes mellitus.

### FIGURES

FIG. 1 Risk of severe hypoglycaemia according to serum ACE activity in type 1 diabetic subjects untreated with ACE inhibitors or angiotensin II receptor antagonists.

Dashed lines show 95% confidence limits (n=207).

FIG. 2 Risk of severe hypoglycaemia according to serum ACE in type 1 diabetic subjects untreated with ACE inhibitors or angiotensin II receptor antagonists in sub-groups according to C-peptide status and level of self-estimated awareness of hypoglycaemia (n=202).

Bold line shows C-peptide negative subjects with impaired awareness (n=47).

Bold dashed line shows C-peptide positive subjects with impaired awareness (n=63).

Thin line shows C-peptide negative subjects with normal awareness (n=32).

Thin dashed line shows C-peptide positive subjects with normal awareness (n=60).

FIG. 3 Risk of severe hypoglycaemia according to HbA1c and quartiles of serum ACE in type 1 diabetic subjects untreated with ACE inhibitors or angiotensin II receptor antagonists (n=207).

Bold line shows fourth quartile.

Thin line shows third quartile.

Thin dashed line shows second quartile.

Bold dashed line shows first quartile.

### DETAILED DESCRIPTION OF THE INVENTION

Amino Acids and Nucleic Acids

Throughout the description and claims the three-letter code for natural amino acids are used. Where the L or D form has not been specified it is to be understood that the amino acid in question has the natural L form, cf. Pure & Appl. Chem. Vol. (56(5) pp 595-624 (1984) or the D form, so that the peptides formed may be constituted of amino acids of L form, D form, or a sequence of mixed L forms and D forms.

Where nothing is specified it is to be understood that the C-terminal amino acid of a polypeptide of the invention exists as the free carboxylic acid, this may also be specified as “—OH”. The N-terminal amino acid of a polypeptide comprise a free amino-group, this may also be specified as “H—”.

Where nothing else is specified amino acid can be selected from any amino acid, whether naturally occurring or not, such as alpha amino acids, beta amino acids, and/or gamma amino acids. Accordingly, the group comprises but are not limited to: Ala, Val, Leu, Ile, Pro, Phe, Trp, Met, Gly, Ser, Thr, Cys, Tyr, Asn, Gln, Asp, Glu, Lys, Arg, His, Aib, Nal, Sar, Orn, Lysine analogues DAP and DAPA.

The term “nucleic acid” is meant to encompass DNA and RNA as well as derivatives thereof such as peptide nucleic acids (PNA) or locked nucleic acids (LNA) through-out the description. When nothing else is stated nucleic acid sequences are indicated starting from the 5′ end. However, usually the position of the 5′ end and 3′ end is indicated in the sequence.

Hybridisation under stringent conditions as used herein shall denote stringency as normally applied in connection with Southern blotting and hybridisation as described e.g. by Southern E. M., 1975, J. Mol. Biol. 98:503-517. For such purposes it is routine practise to include steps of prehybridisation and hybridisation. Such steps are normally performed using solutions containing 6×SSPE, 5% Denhardt's, 0.5% SDS, 50% formamide, 100 μg/ml denaturated salmon testis DNA (incubation for 18 hrs at 42° C.), followed by washings with 2×SSC and 0.5% SDS (at room temperature and at 37° C.), and a washing with 0.1×SSC and 0.5% SDS (incubation at 68° C. for 30 min), as described by Sambrook et al., 1989, in “Molecular Cloning/A Laboratory Manual”, Cold Spring Harbor), which is incorporated herein by reference.

The Renin-angiotensin II System

The term “renin-angiotensin II system” according to the present invention is meant to encompass the process and the components of the process in which angiotensin I is converted to angiotensin II by the angiotensin converting enzyme, as well as the process in which angiotensin II executes its functions.

Accordingly, components of the renin-angiotensin II system according to the present invention may be selected from the group consisting of angiotensin I, angiotensin II, angiotensin converting enzyme and angiotensin II receptors.

Inhibitors of the renin-angiotensin II system may be any substance capable of inhibiting directly or indirectly the conversion of angiotensin I to angiotensin II or any substance capable of inhibit the biological function of angiotensin II, for example any substance that can inhibit the interaction between angiotensin II and angiotensin II receptors or any substance that can inhibit activation of angiotensin II receptors by angiotensin II.

For example, a substance capable of inhibiting the conversion of angiotensin I to angiotensin II could be an inhibitor of angiotensin-converting enzyme (ACE). Such an inhibitor may be a naturally occurring inhibitor or it may be a synthetic inhibitor. In one embodiment of the present invention the inhibitor is a polypeptide, such as for example teprotide.

Preferably, the inhibitor of ACE may be selected from the group consisting of quinapril, lisinopril, enalapril, captopril, benazepril, perindopril, trandolapril, fosinopril, meoxipril, ramipril and teprotide.

A substance capable of inhibiting the biological function of angiotensin II could for example be an angiotensin II-receptor antagonist. Preferably, such an antagonist is capable of associating with the angiotensin II receptor, but is not capable of activating said receptor. Such an antagonist could for example be a polypeptide. In one example an angiotensin II-receptor antagonist may be capable of associating with AT1 receptors, but are not able to induce vasoconstriction and fluid retention upon association.

Preferably, such a polypeptide comprises an amino acid sequence which is similar to the angiotensin II sequence (see herein above), but wherein 1, such as 2, for example 3, such as 4, for example 5, such as 6, for example 7 amino acids have been substituted for an other amino acid. Alternatively, such a polypeptide may be similar to the angiotensin II sequence, but comprise deletions of for example 1, such as 2, for example 3, such as 4, for example 5 amino acids and/or additions of more than 1, such as more than 5, for example more than 10, such as more than 20 amino acids. Polypeptide similar to the angiotensin II sequence may also contain both substitutions and/or deletions and/or additions.

Preferably, the angiotensin II-receptor antagonist selected from the group consisting of candesartancilexetil, eprosartan, irbesartan, losartan, valsartan, telmisartan and saralasin.

Alternatively, the substance capable of inhibiting the biological function of angiotensin II, may be a substance such as for example a polypeptide, capable of interacting with angiotensin II in a way which inhibits the interaction between angiotensin II and its receptor.

Antidiabetics The term “antidiabetic” is meant to encompass any substance or pharmaceutical composition, which can be used for prophylactic, ameliorative or curative treatment of diabetes mellitus, wherein diabetes mellitus may be any type of diabetes mellitus as outlined herein-below.

Suitable antidiabetics comprise insulin, GLP-1 derivatives such as those disclosed in WO 98/08871 (Novo Nordisk A/S), which is incorporated herein by reference as well as orally active hypoglycemic agents.

In one preferred embodiment the antidiabetic is insulin or an analogue thereof or a derivative thereof. More preferably the antidiabetic is human insulin or an analogue thereof or a derivative thereof. However, porcine insulin is also an insulin species, which may be employed with the present invention. Preferably, porcine insulin is highly purified naturally produced porcine insulin.

Human insulin could be naturally produced insulin, preferably however human insulin is recombinantly produced. Recombinant human insulin may be produced in any suitable host cell for example the host cells may be bacterial, fungal (including yeast), insect, animal or plant cells. Preferably, the host cells are yeast cells or bacterial cells such as for example E. coli.

Preferably, the analogue of human insulin is a rapid-acting analogue. For example the analogue may be selected from the group consisting of AspB28 human insulin and LysB28ProB29 human insulin.

In one preferred embodiment the derivative is human insulin or an analogue thereof containing a C₆ to C₄₀ lipophilic substituent in position B29. Preferably, the derivative may be selected from the group consisting of B29-N^{ε}-myristoyl-des(B30) human insulin, B29-N^{ε}-palmitoyl-des(B30) human insulin, B29-N^{ε}-myristoyl human insulin, B29-N^{ε}-palmitoyl human insulin, B28-N^{ε}-myristoyl Lys^{B28}Pro^{B29} human insulin, B28-N^{ε}-palmitoyl Lys^{B28}Pro^{B29} human insulin, B30-N^{ε}-myristoyl-Thr^{B29}Lys^{B30} human insulin, B30-N^{ε}-palmitoyl-Thr^{B29}Lys^{B30} human insulin, B29-N^{ε}-(N-palmitoyl-γ-glutamyl)-des(B30) human insulin, B29-N^{ε}-(N-lithocholyl-γ-glutamyl)-des(B30) human insulin, B29-N^{ε}-(ω-carboxyheptadecanoyl)-des(B30) human insulin and B29-N^{ε}-(ω-carboxyheptadecanoyl) human insulin.

In addition, a variety of different insulin compositions are antidiabetics which should also be considered to fall within the scope of the present invention. For example this includes regular insulin, Semilente® insulin, isophane insulin, insulin zinc suspensions, protamine zinc insulin, and Ultralente® insulin.

Isophane insulin is an isophane mixture of protamine and insulin, wherein a ratio of protamine to insulin is mixed, which is equal to the ratio in a solution made by mixing equal parts of a solution of the two in which all the protamine precipitates and a solution of the two in which all the insulin precipitates.

In one embodiment insulin compositions according to the present invention are characterised by a fast onset of action, while in other embodiments the insulin compositions have a relatively slow onset but show a more or less prolonged action. Fast acting insulin compositions are usually solutions of insulin, while retarded acting insulin compositions can be suspensions containing insulin in crystalline and/or amorphous form precipitated by addition of zinc salts alone or by addition of protamine or by a combination of both. In addition, some compositions have both a fast onset of action and a more prolonged action. Such a composition may be an insulin solution wherein protamine insulin crystals are suspended. Furthermore, compositions obtained by mixing an insulin solution with a suspension composition in the desired ratio are useful with the present invention.

The present invention preferably, may be used in connection with compositions comprising analogues and/or derivatives of human insulin. Thus, the insulin composition according to the invention may comprise one or more fast-acting analogues of human insulin, in particular analogues wherein the amino acid residue at position B28 is Asp, Lys, Leu, Val or Ala and the amino acid residue at position B29 is Lys or Pro; or des(B28-B30), des(B27) or des(B30) human insulin. The insulin analogue is preferably selected from analogues of human insulin wherein the amino acid residue at position B28 is Asp or Lys, and the amino acid residue at position B29 is Lys or Pro. The most preferred analogues are AsP_{B28} human insulin and Lys_{B28}Pro_{B29} human insulin.

In another embodiment the insulin composition according to the invention comprises an insulin derivative having a protracted profile of action, such an insulin having one or more lipophilic substituents. Lipophilic insulins may be acylated insulins, including those described in WO 95/07931, e.g. human insulin derivatives wherein the ε-amino group of Lys_{B29} contains an acyl substituent which comprises at least 6 carbon atoms.

In another embodiment of the present invention the antidiabetic belongs to the group of antidiabetica which can be administrated orally.

For example, the antidiabetic according to the present invention may be an orally active hypoglycemic agent. Orally active hypoglycemic agents preferably comprise sulfonylureas, biguanides, meglitinides, oxadiazolidinediones, thiazolidinediones, α-glucosidase inhibitors, glucagon antagonists such as those disclosed in WO 99/01423 and WO 00/39088 (Novo Nordisk A/S and Agouron Pharmaceuticals, Inc.), GLP-1 agonists such as those disclosed in WO 00/42026 (Novo Nordisk A/S and Agouron Pharmaceuticals, Inc.), potassium channel openers such as those disclosed in WO 97/26265, WO 99/03861 and WO 00/37474 (Novo Nordisk A/S), insulin sensitizers, DPP-IV inhibitors, PTPase inhibitors, inhibitors of hepatic enzymes involved in stimulation of gluconeogenesis and/or glycogenolysis, glucose uptake modulators, compounds modifying the lipid metabolism such as antihyperlipidemic agents and antilipidemic agents, compounds lowering food intake, PPAR and RXR agonists and agents acting on the ATP-dependent potassium channel of the β-cells.

The group of biguanids decreases the blood sugar levels by inhibition of glucose uptake in the intestine, increase of peripheral glucose uptake and inhibition of glucose synthesis in the liver. The group for example comprises metformin.

The group of sulfonylureas stimulates the β-cells of the pancreas to produce more insulin. The group of sulfonylureas for example comprises glibenclamide, glicazide, acetohexamide, chlorpropamide, glimepiride, glipizide, glyburide, tolazamide and tolbutamide.

Alpha-glucosidase inhibitors may for example be selected from the group consisting of acarbose or miglitol.

Meglitinides may for example be selected from the group consisting of repaglinide, nateglinide or senaglinide.

Thiazolidinedione may for example be selected from the group consisting of pioglitazone, rosiglitazone, troglitazone, ciglitazone and the compounds disclosed in WO 97/41097, WO 97/41119, WO 97/41120, WO 00/41121 and WO 98/45202 (Dr. Reddy's Research Foundation).

Insulin sensitizers may for example be those disclosed in WO 99/19313, WO 00/50414, WO 00/63191, WO 00/63192, WO 00/63193 (Dr. Reddy's Research Foundation) and WO 00/23425, WO 00/23415, WO 00/23451, WO 00/23445, WO 00/23417, WO 00/23416, WO 00/63153, WO 00/63196, WO 00/63209, WO 00/63190 and WO 00/63189 (Novo Nordisk A/S).

Agents acting on the ATP-dependent potassium channel of the β-cells may for example be selected from the group consisting of tolbutamide, glibenclamide, glipizide, glicazide and repaglinide.

Preferably the oral antidiabetic is selected from the group consisting of tolbutamid, pioglitazone, rosiglitazone, glibenclamid, gliclazide, glipizide, acarbose, metformin and repaglinide.

Furthermore, the inhibitors of the renin-angiotensin II system or the kit-of-parts according to the present invention may be administered in combination with nateglinide.

Diabetes Mellitus

In one embodiment the present invention relates to preventing hypoglycaemia, preferably severe hypoglycaemia, in an individual in need thereof. Preferably, such an individual is suffering from diabetes mellitus. Hypoglycaemia is a condition of low blood sugar levels, for example a blood sugar level below 4 mmol/l, such as below 3 mmol/l, for example below 2.5 mmol/l, such as below 2 mmol/l. Severe hypoglycaemia is episodes, wherein assistance from other persons is needed to restore normal blood glucose levels

The individuals to be treated according to the present invention are preferably individuals suffering from diabetes mellitus. Diabetes mellitus is a syndrome characterised by insufficient insulin secretion or reduced ability of the body's cells to utilise insulin resulting in reduced glucose tolerance. Diabetes mellitus can be classified into different types and the basic types, type 1 and type 2. Type 1 diabetes usually occurs in children and adults under age 30. Type 2 diabetes is a result of progressive dysfunction of the beta cells and the reduced ability of the body's cells to use insulin, known as insulin resistance.

Diabetes mellitus according to the present invention includes any type of diabetes mellitus, for example diabetes mellitus type 1 and diabetes mellitus type 2 should be considered to fall within the scope of the present invention. Preferably, the diabetes mellitus is diabetes mellitus type 1.

The treatment according to the present invention may be prophylactic treatment, it may be curative treatment or it may be ameliorating treatment.

The individual according to the present invention could be any animal, preferably however the individual is a human being, more preferably a human being suffering from diabetes mellitus.

Pharmaceutical Compositions and Administration Forms

The route of administration to be used with the present invention may be any administration form suitable for the particular embodiment of the invention.

The main routes of drug delivery according to the present invention are intravenous, subcutaneous, oral, and topical, as will be described below. Other drug-administration methods, which are effective to deliver the drug to a target site or to introduce the drug into the bloodstream, are also contemplated.

The mucosal membrane to which the compounds of the invention are administered may be any mucosal membrane of the mammal to which the biologically active substance is to be given, e.g. in the nose, vagina, eye, mouth, genital tract, lungs, gastrointestinal tract, or rectum.

Compounds of the invention may be administered parenterally, that is by intravenous, intramuscular, subcutaneous intranasal, intrarectal, intravaginal or intraperitoneal administration. Preferably, parenteral administration is by subcutaneous injection. Appropriate dosage forms for such administration may be prepared by conventional techniques.

The compounds may also be administered orally or by inhalation, that is by intranasal and oral inhalation administration. Preferably, the administration is oral administration.

More preferably, the compounds according to the present invention are administrated either orally or by subcutaneous injection.

In one embodiment of the present invention the inhibitors of the renin-angiotensin II system are administrated in a manner to obtain the best possible bioavailability of these compounds in the body compartments wherein the effect of ACE on severe hypoglycaemia is exerted. For example such a body compartment may be the CNS.

The kit-of-parts according to the present invention may furthermore be administrated in a manner, such as one or more components of the kit-of-parts are administrated by one route and another one or more components of the kit-of-parts are administrated by another route. By way of example, one component may be administrated orally, whereas another component may be administrated by subcutaneous injection.

Furthermore, the individual compounds of the kit-of-parts according to the present invention may be administered simultaneously, either as separate formulations or combined in a unit dosage form, or they may be administered sequentially.

The compounds according to the invention may also be administered with at least one additional compound.

The dosage requirements will vary with the particular drug composition employed, the route of administration and the particular individual being treated. Ideally, an individual to be treated by the present method will receive a pharmaceutically effective amount of the compound in the maximum tolerated dose, generally no higher than that required before drug resistance develops.

For all methods of use disclosed herein for the compounds, the daily oral dosage regimen will preferably be from about 0.01 to about 80 mg/kg of total body weight. The daily parenteral dosage regimen about 0.001 to about 80 mg/kg of total body weight. The daily topical dosage regimen will preferably be from 0.1 mg to 150 mg, administered one to four, preferably two or three times daily. The daily inhalation dosage regimen will preferably be from about 0.01 mg/kg to about 1 mg/kg per day. It will also be recognized by one of skill in the art that the optimal quantity and spacing of individual dosages of a compound or a pharmaceutically acceptable salt thereof will be determined by the nature and extent of the condition being treated, the form, route and site of administration, and the particular patient being treated, and that such optimums can be determined by conventional techniques. It will also be appreciated by one of skill in the art that the optimal course of treatment, i.e., the number of doses of a compound or a pharmaceutically acceptable salt thereof given per day for a defined number of days, can be ascertained by those skilled in the art using conventional course of treatment determination tests.

The term “unit dosage form” as used herein refers to physically discrete units suitable as unitary dosages for human and animal individuals, each unit containing a predetermined quantity of a compound, alone or in combination with other agents, calculated in an amount sufficient to produce the desired effect in association with a pharmaceutically acceptable diluent, carrier, or vehicle. The specifications for the unit dosage forms of the present invention depend on the particular compound or compounds employed and the effect to be achieved, as well as the pharmacodynamics associated with each compound in the host. The dose administered should be an “effective amount” or an amount necessary to achieve an “effective level” in the individual patient.

Since the “effective level” is used as the preferred endpoint for dosing, the actual dose and schedule can vary, depending on interindividual differences in pharmacokinetics, drug distribution, and metabolism. The “effective level” can be defined, for example, as the blood or tissue level desired in the individual that corresponds to a concentration of one or more compounds according to the invention.

Pharmaceutical compositions containing a compound of the present invention may be prepared by conventional techniques, e.g. as described in Remington: The Science and Practice of Pharmacy 1995, edited by E. W. Martin, Mack Publishing Company, 19th edition, Easton, Pa. The compositions may appear in conventional forms, for example capsules, tablets, aerosols, solutions, suspensions or topical applications.

Pharmaceutical acceptable salts of the compounds according to the present invention should also be considered to fall within the scope of the present invention. Pharmaceutically acceptable salts are prepared in a standard manner. If the parent compound is a base it is treated with an excess of an organic or inorganic acid in a suitable solvent. If the parent compound is an acid, it is treated with an inorganic or organic base in a suitable solvent.

The compounds of the invention may be administered in the form of an alkali metal or earth alkali metal salt thereof, concurrently, simultaneously, or together with a pharmaceutically acceptable carrier or diluent, especially and preferably in the form of a pharmaceutical composition thereof, whether by oral, rectal, or parenteral (including subcutaneous) route, in an effective amount.

Examples of pharmaceutically acceptable acid addition salts for use in the present inventive pharmaceutical composition include those derived from mineral acids, such as hydrochloric, hydrobromic, phosphoric, metaphosphoric, nitric and sulfuric acids, and organic acids, such as tartaric, acetic, citric, malic, lactic, fumaric, benzoic, glycolic, gluconic, succinic, p-toluenesulphonic acids, and arylsulphonic, for example.

Whilst it is possible for the compounds or salts of the present invention to be administered as the raw chemical, it is preferred to present them in the form of a pharmaceutical formulation. Accordingly, the present invention further provides a pharmaceutical formulation, for medicinal application, which comprises a compound of the present invention or a pharmaceutically acceptable salt thereof, as herein defined, and a pharmaceutically acceptable carrier therefor.

The compounds of the present invention may be formulated in a wide variety of oral administration dosage forms. The pharmaceutical compositions and dosage forms may comprise the compounds of the invention or its pharmaceutically acceptable salt or a crystal form thereof as the active component. The pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, wetting agents, tablet disintegrating agents, or an encapsulating material.

Preferably, the composition will be about 0.5% to 75% by weight of the compounds of the invention, with the remainder consisting of suitable pharmaceutical excipients. For oral administration, such excipients include pharmaceutical grades of mannitol, lactose, starch, magnesium stearate, sodium saccharine, talcum, cellulose, glucose, gelatin, sucrose, magnesium carbonate, and the like.

In powders, the carrier is a finely divided solid, which is a mixture with the finely divided active component. In tablets, the active component is mixed with the carrier having the necessary binding capacity in suitable proportions and compacted in the shape and size desired. The powders and tablets preferably contain from one to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is in association with it. Similarly, cachets and lozenges are included. Tablets, powders, capsules, pills, cachets, and lozenges can be as solid forms suitable for oral administration.

Drops according to the present invention may comprise sterile or non-sterile aqueous or oil solutions or suspensions, and may be prepared by dissolving the active ingredient in a suitable aqueous solution, optionally including a bactericidal and/or fungicidal agent and/or any other suitable preservative, and optionally including a surface active agent. The resulting solution may then be clarified by filtration, transferred to a suitable container which is then sealed and sterilized by autoclaving or maintaining at 98-100° C. for half an hour. Alternatively, the solution may be sterilised by filtration and transferred to the container aseptically. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Other forms suitable for oral administration include liquid form preparations including emulsions, syrups, elixirs, aqueous solutions, aqueous suspensions, toothpaste, gel dentrifrice, chewing gum, or solid form preparations which are intended to be converted shortly before use to liquid form preparations. Emulsions may be prepared in solutions in aqueous propylene glycol solutions or may contain emulsifying agents such as lecithin, sorbitan monooleate, or acacia. Aqueous solutions can be prepared by dissolving the active component in water and adding suitable colorants, flavors, stabilizing and thickening agents. Aqueous suspensions can be prepared by dispersing the finely divided active component in water with viscous material, such as natural or synthetic gums, resins, methylcellulose, sodium carboxymethylcellulose, and other well known suspending agents. Solid form preparations include solutions, suspensions, and emulsions, and may contain, in addition to the active component, colorants, flavors, stabilizers, buffers, artificial and natural sweeteners, dispersants, thickeners, solubilizing agents, and the like.

The compounds of the present invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilisation from solution for constitution before use with a suitable vehicle, e.g., sterile, pyrogen-free water.

Oils useful in parenteral formulations include petroleum, animal, vegetable, or synthetic oils. Specific examples of oils useful in such formulations include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides; (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-.beta.-aminopropionates, and 2-alkyl-imidazoline quaternary ammonium salts, and (e) mixtures thereof.

The parenteral formulations typically will contain from about 0.5 to about 25% by weight of the active ingredient in solution. Preservatives and buffers may be used. In order to minimize or eliminate irritation at the site of injection, such compositions may contain one or more nonionic surfactants having a hydrophilic-lipophilic balance (HLB) of from about 12 to about 17. The quantity of surfactant in such formulations will typically range from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

The compounds of the invention can also be delivered topically. Regions for topical administration include the skin surface and also mucous membrane tissues of the vagina, rectum, nose, mouth, and throat. Compositions for topical administration via the skin and mucous membranes should not give rise to signs of irritation, such as swelling or redness.

The topical composition may include a pharmaceutically acceptable carrier adapted for topical administration. Thus, the composition may take the form of a suspension, solution, ointment, lotion, sexual lubricant, cream, foam, aerosol, spray, suppository, implant, inhalant, tablet, capsule, dry powder, syrup, balm or lozenge, for example. Methods for preparing such compositions are well known in the pharmaceutical industry.

The compounds of the present invention may be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

When desired, formulations can be prepared with enteric coatings adapted for sustained or controlled release administration of the active ingredient.

Pharmaceutical compositions usually comprise a carrier. Illustrative solid carrier include lactose, terra alba, sucrose, talc, gelatin, agar, pectin, acacia, magnesium stearate, stearic acid and the like. A solid carrier can include one or more substances which may also act as flavoring agents, lubricants, solubilizers, suspending agents, fillers, glidants, compression aids, binders or tablet-disintegrating agents; it can also be an encapsulating material. In powders, the carrier is a finely divided solid which is in admixture with the finely divided active ingredient. In tablets, the active ingredient is mixed with a carrier having the necessary compression properties in suitable proportions, and compacted in the shape and size desired. The powders and tablets preferably contain up to 99% of the active ingredient. Suitable solid carriers include, for example, calcium phosphate, magnesium stearate, talc, sugars, lactose, dextrin, starch, gelatin, cellulose, methyl cellulose, sodium carboxymethyl cellulose, polyvinylpyrrolidine, low melting waxes and ion exchange resins.

Illustrative liquid carriers include syrup, peanut oil, olive oil, water, etc. Liquid carriers are used in preparing solutions, suspensions, emulsions, syrups, elixirs and pressurized compositions. The active ingredient can be dissolved or suspended in a pharmaceutically acceptable liquid carrier such as water, an organic solvent, a mixture of both or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators. Suitable examples of liquid carriers for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also be an oily ester such as ethyl oleate and isopropyl myristate. Sterile liquid carders are useful in sterile liquid form compositions for parenteral administration. The liquid carrier for pressurized compositions can be halogenated hydrocarbon or other pharmaceutically acceptable propellant. Liquid pharmaceutical compositions which are sterile solutions or suspensions can be utilized by, for example, intramuscular, intraperitoneal or subcutaneous injection. Sterile solutions can also be administered intravenously. The compound can also be administered orally either in liquid or solid composition form.

The carrier or excipient may include time delay material well known to the art, such as glyceryl monostearate or glyceryl distearate along or with a wax, ethylcellulose, hydroxypropylmethylcellulose, methylmethacrylate and the like. When formulated for oral administration, 0.01% Tween 80 in PHOSAL PG-50 (phospholipid concentrate with 1,2-propylene glycol, A. Nattermann & Cie. GmbH) has been recognized as providing an acceptable oral formulation for other compounds, and may be adapted to formulations for various compounds of this invention.

Diagnosis

The present invention is also concerned with methods to diagnose the susceptibility to hypoglycaemia of an individual. The first step in such a diagnosis is to obtain a tissue sample from said individual.

The tissue sample may be any tissue sample suitable for the embodiment of the invention, however for most purposes the tissue sample is preferably a blood sample.

For specific embodiments of the present invention it is preferred that said blood sample is further purified to obtain serum.

In one embodiment of the present invention the diagnosis is based on determining the ACE genotype of the individual. Preferably, the ACE genotype is selected from the group consisting of II, ID or DD, wherein I designates the insertion allele and D designates the deletion allele of the ACE gene.

The difference between the I and the D allele of the ACE gene corresponds to an insertion/deletion of 288 base pairs (SEQ ID. 2) in intron 16 of the human ACE gene.

The sequence of intron 16 of the I allele is depicted in SEQ ID. 1.

The sequence of intron 16 of the D allele corresponds to SEQ ID. 1, from which base pairs 1451 to 1738 have been deleted.

Generally, the II genotype correlates with low susceptibility, the ID genotype correlates with medium susceptibility and the DD genotype correlates with high susceptibility to hypoglycaemia.

The genotype may be determined by any suitable method known to the person skilled in the art. For example the method may be a nucleic acid techniques based on hybridisation, size, or sequence, such as restriction fragment length polymorphism (RFLP), nucleic acid sequencing or fluorescent in situ hybridisation (FISH).

These methods may also comprise the step of amplifying the nucleic acid before analysis. Amplification techniques are known to those of skill in the art and include cloning, polymerase chain reaction (PCR), polymerase chain reaction of specific alleles (PASA), polymerase chain ligation, nested polymerase chain reaction, Single-Stranded Conformational Polymorphism (SSCP) analysis and the like. Amplification products may be assayed in a variety of ways, including size analysis, restriction digestion followed by size analysis, detecting specific tagged oligonucleotide primers in the reaction products, allele-specific oligonucleotide (ASO) hybridisation, sequencing, and the like.

Alternatively, allele detection techniques may be protein based, for example, epitopes specific for the amino acid variant produced from the I and the D allele can be detected with specific monoclonal antibodies.

In one preferred embodiment the method comprises the steps of

a) purifying genomic DNA from a tissue sample; and

b) amplifying said DNA by polymerase chain reaction (PCR) using at least one pair of primers consisting of a first primer and a second primer, wherein said first primer and said second primer are specific for the ACE gene; and

c) analysing the PCR products such as the ACE genotype can be determined.

Preferably, the first primer comprises a nucleotide sequence derived from the ACE gene sequence, more preferably from the sequence of intron 16 of the ACE gene (SEQ. ID. NO: 1). The first primer preferably consists of 10 to 15 nucleotides, such as 15 to 20 nucleotide, such as 20 to 25 nucleotides, such as 25 to 30 nucleotides, such as 30 to 35 nucleotides, such as 35 to 40 nucleotides, such as more than 40 nucleotides.

In one embodiment said first primer comprises the nucleotide sequence

5′ CTG GAG ACC ACT CCC ATC CTT TCT 3′

Preferably, the second primer comprises a nucleotide sequence capable of hybridising under stringent conditions to a sequence derived from the ACE gene sequence, for example a sequence which may hybridise to the sequence of intron 16 of the ACE gene (SEQ. ID: NO.: 1). The second primer consists of 10 to 15 nucleotides, such as 15 to 20 nucleotide, such as 20 to 25 nucleotides, such as 25 to 30 nucleotides, such as 30 to 35 nucleotides, such as 35 to 40 nucleotides, such as more than 40 nucleotides.

In one embodiment the second primer comprise the nucleotide sequence

5′ GAT GTG GCC ATC ACA TTC GTC AGA T 3′

In one embodiment of the present invention more than one pair of primers are used, such as genomic DNA from one tissue sample is subjected to more than one different PCRs, each PCR comprising the use of a specific pair of primers. This could for example be 2 different, such as 3 different, for example 4 different, such as 5 different, for example more than 5 different PCRs. Preferably, at least two pairs of primers are used.

The first primer of the second set of primers may be selected as outlined herein above for the first primer. In one preferred embodiment however the first primer of the second pair of primers comprises a nucleotide sequence derived from the ACE insertion (SEQ ID NO. 2).

An example of such a primer is:

5′ TGG GAC CAC AGC GCC CGC CAC TAC 3′

The second primer of the second set of primers may be selected as outlined herein above for the second primer.

An example of such a primer is

5′ TCG CCA GCC CTC CCA TGC CCA TAA 3′

In one preferred embodiment, however, the second primer of the second pair of primers comprises a nucleotide sequence capable of hybridising under stringent conditions to a sequence derived from the nucleotide sequence of the ACE insertion (SEQ. ID. 2).

Alternatively, genomic DNA may be subjected to one first PCR and subsequently the product of that first PCR is subjected to one second PCR and so forth. In such an embodiment of the invention the second pair of primers should be selected such as they can hybridise with the product of the first PCR.

The primers according to the present invention may comprise one or more mutations compared to the ACE gene sequence or the sequence of the complementary strand of the ACE gene such as one nucleotide is exchanged for another nucleotide. For example the primer may comprise 1 mutation, for example 2 mutations, such as 3 mutations, for example 4 mutations, such as 5 mutations, for example 6 mutations, such as 7 mutations, for example 8 mutations, such as 9 mutations, for example 10 mutations, such as more than 10 mutations. The primers should be able to hybridise with the ACE gene or the complementary strand of the ACE gene under the conditions used for the PCR reaction and may therefore only contain so many mutations that hybridisation can still occur.

The primers may also contain deletion and/or addition of nucleotides. For example that may be deletion and/or addition of at least 1 nucleotide, such as at least 2 nucleotides, for example at least 3 nucleotides, such as from 2 to 5 nucleotides, for example 5 to 10 nucleotides, such as from 10 to 15 nucleotides, for example from 15 to 20 nucleotides, such as from 20 to 30 nucleotides, for example more than 30 nucleotides.

The PCR may be performed according to any standard procedure known to the person skilled in the art as for example described in Sambrook et al., (1989) Molecular Cloning: A Laboratory Manual. Cold Spring Harbor Laboratory. The PCR conditions should be optimised according to the individual embodiment of the present invention.

The PCR products should be analysed such, as the ACE genotype can be determined. Any suitable method known to the person skilled in the art may be employed. Such an analysis may for example be size analysis by gel electrophoresis or restriction digestion followed by size analysis.

In another embodiment of the present invention the diagnosis is based on determining the activity of ACE in the tissue sample. Preferably, the tissue sample is a blood sample, more preferably said blood sample is further purified to obtain serum.

The function of ACE is to convert angiotensin I to angiotensin II (see herein above). Accordingly, the activity of ACE can be determined by any assay which can determine the rate of conversion of angiotensin I to angiotensin II. However, other kinds of ACE activity assays than do not measure conversion of angiotensin I to angiotensin II are also available. Any suitable assay known to the person skilled in the art may be used with the present invention. For example the assay may comprise the use of specific immunoreactive species, such as for example a radioimmunoassay or a competitive enzyme-linked immunoassay or the assay may for example be a spectrophotometric assay. Preferably, the activity of ACE is determined from a serum sample using a kinetic assay.

In one embodiment the assay is a spectrophotometric assay utilising FAPGG as substrate. FAPGG is a synthetic tripeptide substrate (N-[3-(2-furyl)acryloyl]-L-phenylalanylglycylglycine). The hydrolysis of FAPGG to produce FAP (furylacryloylphenylalanine) and glycylglycine is catalysed by ACE and this hydrolysis results in a decrease in absorbance at 340 nm. Accordingly, the activity of ACE can be correlated to the absorbance measured at 340 nm.

Generally, high ACE activity is correlated with high susceptibility to hypoglycaemia and low ACE activity is correlated with low susceptibility to hypoglycaemia. Preferably, the risk of hypoglycaemia is increased 1.1 to 2.0, preferably 1.1 to 1.7, more preferably 1.2 to 1.6 times per 10 U/l increment in serum ACE activity.

In one embodiment of the present invention one or more other parameters are furthermore determined. In particular, parameters, which are known principal determinants of hypoglycaemia may be determined. Preferably, one or more parameters selected from the group consisting of C-peptide concentration and haemoglobin A concentration may furthermore be determined.

In one embodiment of the present invention diagnosis of susceptibility to hypoglycaemia in an individual is performed in order determine an appropriate dose of an antidiabetic to treat said individual.

In particular, an individual, which has high susceptibility to hypoglycaemia, should preferably be treated with inhibitors of the renin-angiotensin II system, whereas individuals with low susceptibility to hypoglycaemia, may not need treatment with inhibitors of the renin-angiotensin II system.

An individual which has a high susceptibility to hypoglycaemia, is for example an individual having the DD ACE genotype and/or an individual which has serum ACE activity levels higher than 60 U/l, preferably higher than 70 U/l, more preferably higher than 75 U/l, yet more preferably higher than 80 U/l, even more preferably higher than 85 U/l, most preferably higher than 90 U/l.

An individual which has a low susceptibility to hypoglycaemia, is for example am individual having the II ACE genotype and/or an individual which has serum ACE activity levels lower than 70 U/l, preferably lower than 60 U/l, more preferably lower than 55 U/l, yet more preferably lower than 50 U/l.

### EXAMPLES

Methods

Subjects

Consecutive adult (>18 years of age) outpatients with clinical type 1 diabetes mellitus for more than two years, who were not on haemodialysis, pregnant or suffering from concomitant malignant disease were included in the study. Type 1 diabetes was defined by insulin treatment from the time of diagnosis and unstimulated C-peptide<300 pmol/l or stimulated (venous blood glucose concentration>12 mmol/l) C-peptide<600 pmol/l. The study comprised 207 subjects untreated with ACE inhibitors or angiotensin II receptor antagonists. The clinical characteristics of the non-participants (not shown) did not differ from the participating subjects (Table 1).

The study was approved by the regional Ethics Committee and written informed consent was obtained from all participants.

Questionnaire

The subjects at home filled in a questionnaire that, at their first subsequent visit in the outpatient clinic at Hillerød Hospital, was collected and checked by the research nurse together with the patient in order to ensure a common understanding of the questions and definitions used. The questionnaire was a brief version of that previously used in our British-Danish multi-centre survey of hypoglycaemia and awareness of hypoglycaemia (12), based on a questionnaire originally developed by Pramming et al (13). Mild hypoglycaemic episodes were reported for the previous week and were defined as subjective symptoms of hypoglycaemia, manageable by the patient. Severe hypoglycaemic episodes were defined as episodes, where assistance from other persons was needed to restore normal blood glucose levels. These episodes were reported for the preceding one-year and two-year periods in order to evaluate a possible loss of recall over time. The two-year report that has been used previously by others (5,14) was selected as primary endpoint as it offers an expression of the chronic risk of severe hypoglycaemia and, as opposed to the number of episodes during lifetime, is independent of diabetes duration. Awareness of hypoglycaemia was scored by the patients on a four-point scale ranging from ‘never’ to ‘always’ to the question: “Can you feel when you are low?”. Subjects giving answers apart from ‘always’ were classified as having impaired awareness. A question of similar character has been used by a number of other groups (15,16), either alone or as part of scoring systems, for classification of awareness level.

Clinical Data

Data on the history of diabetes and its late complications were extracted from the patients' medical records. Existing late complications were graded as follows: a) retinopathy: untreated or laser treated; b) nephropathy: microalbuminuria (urinary albumin excretion rate 30 to 300 mg per 24 hours) or diabetic nephropathy (urinary albumin excretion rate>300 mg per 24 hours); c) peripheral neuropathy: asymptomatic (defined by elevated age-standardised vibration sense threshold at biothesiometry or absence of Achilles tendon reflexes on both feet but no subjective symptoms) or symptomatic (defined by presence of a clinical symptom); d) autonomic neuropathy: asymptomatic (decreased beat to beat variation or orthostatic hypotension but no subjective symptoms) or symptomatic (presence of a symptom of autonomic dysfunction).

Laboratory Analyses

ACE genotype was determined on total genomic DNA extracted from frozen whole blood by polymerase chain reaction (PCR), including a check for misclassification of DD subjects using an insertion specific primer as previously described (17). ACE activity in serum was determined by an assay based on kinetics (Sigma Diagnostics, St. Louis, Mo., USA). The intra- and inter-assay variability of the method was 13% and 11%, respectively. C-peptide was determined by a radioimmunoassay (AutoDELFIA, Wallac Oy, Turku, Finland) and subjects were classified as being without residual beta cell function if C-peptide was below the detection limit of 10 pmol/l. HbA1c was measured spectrophotometrically by a DCA-2000 (Bayer, Leverkusen, Germany) standardised against DCCT (normal range 4.1% to 6.4%).

Data from questionnaires and medical records were collected blind to the results of biochemical analyses and vice versa.

Statistical Analysis

Primary endpoint was self-reported frequency of severe hypoglycaemia during the preceding two-year period. Secondary parameters were episodes of mild hypoglycaemia during the previous week, awareness of hypoglycaemia and residual beta cell function.

Standard descriptive and non-parametric comparative statistics were used to characterise and compare groups. The number of severe and mild hypoglycaemic episodes were analysed by a frailty model for recurrent events (an extension of the log-linear Poisson model including a gamma-distributed variation between patients) (18) and the effect of an explanatory factor was reported as the relative risk (RR) with 95% confidence limits when the factor was present. Data were processed by use of the SPSS software package and a non-commercial software program. The level of statistical significance was chosen as <0.05.

Results

The clinical characteristics of the 207 participants are shown in Table 1.

TABLE 1
Clinical characteristics of 207 type 1 diabetic patients. Values are mean
(SD) or percentage when indicated.

| | No ACE inhibitor or ATII receptor |
|---|---|
| | antagonist treatment (n = 207) |
| Age (years) | 43.1 (12.8) |
| Gender (% female) | 45.6 |
| BMI (kg/m²) | 25.0 (3.7) |
| Duration of diabetes (years) | 18.4 (10.9) |
| C-peptide (undetectable/low (%))* | 39.0/61.0 |
| HbA1c (%) | 8.6 (1.27) |
| Retinopathy (%) | 46.0 |
| Nephropathy (%) | 9.8 |
| Peripheral neuropathy (%) | 25.7 |
| Autonomic neuropathy (%) | 9.2 |
| Hypertension (%) | 7.3 |
| Macrovascular complications (%) | 3.4 |
| ≧4 insulin injections per day (%) | 85.4 |
| Daily insulin dose (IU) | 52.4 (19.9) |
| ACE genotype (II/ID/DD) | 23.7/52.2/24.1 |
| Serum ACE (U/I) | 46.7 (16.8) |
| Normal awareness of hypoglycaemia | 45.1 |
| (%) | |

*: undetectable: <10 pmol/l
low: <300 pmol/l (unstimulated) or <600 pmol/l (stimulated)

Compared to patients not treated with ACE inhibitors or angiotensin II receptor antagonists:

†: p<0.001 (Mann-Whitney test)

†: p=0.005 (Pearson's Chi-squared test)

§: p<0.001 (Pearson's Chi-squared test)

The ACE genotype distribution of the 207 subjects (II: 23.7%, ID: 52.2% and DD: 24.1%) was in Hardy-Weinberg equilibrium and was similar to the genotype distribution of a large sample of the Danish general population (17). There was no significant difference between the genotypes with regard to diabetes duration, age of onset, daily insulin dose, frequency of late complications, C-peptide negativity, HbA1c or level of hypoglycaemia awareness (Table 2). A significant correlation between ACE genotype and serum ACE was present with a two-fold increased serum ACE in the DD group compared to the II group (p<0.001) (Table 2).

TABLE 2
Clinical characteristics according to ACE genotype of 207 type 1 diabetic
patients, untreated with ACE inhibitors or ATII receptor antagonists.
Values are mean (SD) or percentage when indicated.

| | II | ID | DD |
|---|---|---|---|
| ACE genotype | (n = 49) | (n = 108) | (n = 50) |
| Age (years) | 43.2 (13.6) | 43.2 (12.7) | 43.0 (12.3) |
| Gender (% female) | 54.2 | 43.5 | 42.0 |
| BMI (kg/m²) | 24.9 (3.4) | 25.0 (3.9) | 25.1 (3.5) |
| Duration of diabetes | 19.2 (10.4) | 18.7 (11.0) | 17.0 (11.3) |
| (years) | | | |
| C-peptide (undetect- | 44.9/55.1 | 34.6/65.4 | 42.9/57.1 |
| able/low (%))* | | | |
| HbA1c (%) | 8.5 (1.41) | 8.6 (1.21) | 8.5 (1.28) |
| Retinopathy (%) | 43.8 | 48.1 | 43.5 |
| Nephropathy (%) | 8.7 | 9.9 | 10.7 |
| Peripheral neuropathy | 22.9 | 29.0 | 21.3 |
| (%) | | | |
| Autonomic neuropathy | 14.8 | 9.5 | 3.4 |
| (%) | | | |
| Hypertension (%) | 8.3 | 7.4 | 6.0 |
| Macrovascular complica- | 2.0 | 5.6 | 0 |
| tions (%) | | | |
| ≧4 insulin injections per | 83.3 | 84.3 | 90.0 |
| day (%) | | | |
| Daily insulin dose (IU) | 51.8 (20.7) | 52.7 (20.2) | 52.1 (18.7) |
| Serum ACE (U/I) | 32.7 (8.1) | 46.7 (12.9) | 60.3 (19.4)† |
| Normal awareness of | 45.8 | 47.2 | 40.0 |
| hypoglycaemia (%) | | | |

*: undetectable: <10 pmol/l
low: <300 pmol/l (unstimulated) or <600 pmol/l (stimulated)
†: p < 0.001 (Kruskal-Wallis test)

### Univariate Analyses of Severe Hypoglycaemia in Subjects Untreated with ACE Inhibitors

ACE Genotype and Severe Hypoglycaemia

The relative risk (RR) of severe hypoglycaemia during the preceding two-year period was 1.5 for the ID genotype (p=0.29) and 3.2 for the DD genotype (p<0.01), respectively, compared to the II subjects (Table 3). This was primarily due to a higher number of episodes per subject with DD genotype reporting severe hypoglycaemia, whereas the proportion of subjects reporting severe hypoglycaemia was not significantly higher in the DD group (II 40.8%, ID 42.6%, DD: 54.0%; p=0.33, Pearson's Chi-squared test).

Table 3 Result of univariate analyses of the influence of ACE genotype, serum ACE and other factors on the risk of severe hypoglycaemia in 207 type 1 diabetic patients untreated with ACE inhibitors and angiotensin II receptor antagonists. The table shows RR with 95% confidence limits and corresponding p-values. RR values represent: ID and DD genotype: RR compared to II genotype; serum ACE: RR per ten unit increment in serum ACE; awareness: RR of subjects with impaired awareness compared to those with normal awareness; C-peptide status: RR of C-peptide negative patients compared to C-peptide positive subjects; HbA1c: RR per one percent increase in HbA1c; age: RR per one year increment; duration: RR compared to those with less than 10 years duration of diabetes.

| Variable | RR (95% CL) | p |
|---|---|---|
| ID genotype | 1.5 (0.7-3.1) | 0.29 |
| DD genotype | 3.2 (1.4-7.4) | <0.01 |
| Serum ACE | 1.4 (1.2-1.6) | <0.001 |
| Awareness | 8.5 (4.8-14.9) | <0.0001 |
| C-peptide status | 2.8 (1.6-5.0) | <0.001 |
| HbA1c | 0.65 (0.51-0.82) | <0.001 |
| Age | 1.028 (1.003-1.054) | <0.05 |
| Duration of diabetes 10-20 years | 1.6 (0.8-3.2) | 0.23 |
| Duration of diabetes >20 years | 3.9 (1.9-7.9) | <0.001 |

Serum ACE and Severe Hypoglycaemia

A significant relation between serum ACE and the rate of severe hypoglycaemia was observed (FIG. 1 and Table 3) with a RR of 1.4 per ten unit increment in serum ACE (p<0.001). This corresponds to a 3.5-fold increased risk of severe hypoglycaemia among subjects with serum ACE in the upper quartile when compared to the lowest quartile. The fraction of subjects reporting severe hypoglycaemia tended to be higher in the upper quartile compared to the lower quartiles (35%, 45%, 41%, 59%, respectively; p=0.10, Pearson's Chi-squared test).

Other Parameters and Severe Hypoglycaemia

The results of univariate analyses of the relation between known risk factors and the risk of severe hypoglycaemia are shown in Table 3. The RR of severe hypoglycaemia in subjects with impaired awareness of hypoglycaemia was 8.5 when compared to patients with normal awareness (p<0.0001). Subjects with undetectable residual beta cell function had a RR of severe hypoglycaemia of 2.8 as compared to those with preserved residual beta cell function (p<0.001). HbA1c levels were negatively related to the risk of severe hypoglycaemia with a RR of 0.65 per one percent increment (p<0.001). Age influenced on the risk of severe hypoglycaemia with a RR of 1.028 per one-year increment (p<0.05). Subjects with diabetes for more than 20 years had a RR of severe hypoglycaemia of 3.9 when compared to patients with less than 10 years of disease (p<0.001).

### Multivariate Analysis of Severe Hypoglycaemia in Subjects Untreated with ACE Inhibitors

Serum ACE and ACE Genotype

The relative importance of serum ACE and the ACE genotype was assessed in a multivariate analysis resulting in a highly significant relation between serum ACE and the risk of severe hypoglycaemia with a RR of 1.5 per ten units of serum ACE (p<0.001). In contrast, the DD genotype was not related to the risk of severe hypoglycaemia (RR 0.7 (0.3-2.0), p=0.53) when the serum ACE was fixed. This indicates that the effect of the genotype was mediated by the phenotypically expressed serum ACE activity. Consequently, only serum ACE is included in the further analyses.

Serum ACE and Other Risk Factors of Severe Hypoglycaemia

To evaluate the relative impact of serum ACE and known markers of severe hypoglycaemia a stepwise regression analysis including serum ACE, hypoglycaemia awareness, beta cell function, HbA1c, age and duration of diabetes was performed (Table 4a). After exclusion of non-significant parameters a significant relation between serum ACE and the rate of severe hypoglycaemia was observed with a RR of 1.3 per ten unit increment of serum ACE. Impaired awareness was associated with a RR of 5.2 compared to normal awareness and undetectable C-peptide of 1.8 compared to detectable residual beta cell function. Duration of diabetes for more than 20 years was associated with a RR of 2.0 compared to duration of less than 20 years. HbA1c and age were not significantly related to severe hypoglycaemia in this model.

Since self-estimated level of awareness of hypoglycaemia is a soft parameter, potentially biased by the previously experienced rate of severe hypoglycaemia, the same analysis was performed following omission of awareness level (Table 4b). This tended to increase the impact of ACE activity and duration of diabetes and included HbA1c as a significant determinant of severe hypoglycaemia with a RR of 0.7 per one percent increment in HbA1c, whereas the effect of C-peptide status did not reach significance (p=0.06).

TABLE 4a
Result of stepwise regression analysis of the influence of serum ACE,
awareness, C-peptide status, duration of diabetes, age and HbA1c on the
risk of severe hypoglycaemia in 207 type 1 diabetic patients untreated with
ACE inhibitors and angiotensin II receptor antagonists. The table shows
RR with 95% confidence limits and corresponding p-values. RR values
represent: serum ACE: RR per ten unit increment in serum ACE;
awareness: RR of subjects with impaired awareness compared to those
with normal awareness; C-peptide status: RR of C-peptide negative
patients compared to C-peptide positive subjects; duration: RR of
patients with diabetes for more than 20 years compared to those with
less than 20 years.

| Variable | RR (95% CL) | p |
|---|---|---|
| Serum ACE | 1.3 (1.1-1.5) | <0.001 |
| Awareness | 5.2 (3.1-9.0) | <0.0001 |
| C-peptide status | 1.8 (1.1-2.9) | <0.05 |
| Duration of diabetes | 2.0 (1.2-3.3) | <0.01 |

TABLE 4b
Result of the same stepwise regression analysis as in Table 4a following
omission of awareness. HbA1c: RR per one per cent increment in HbA1c.

| Variable | RR (95% CL) | p |
|---|---|---|
| Serum ACE | 1.4 (1.2-1.6) | <0.0001 |
| Duration of diabetes | 2.2 (1.3-3.8) | <0.01 |
| HbA1c | 0.7 (0.6-0.9) | <0.01 |

The interplay between serum ACE, C-peptide status and awareness level on the risk of severe hypoglycaemia is illustrated in FIG. 2. The relation between serum ACE and severe hypoglycaemia was significantly stronger in the group of C-peptide negative patients with impaired awareness (RR: 1.7 (1.3-2.1) per ten units of serum ACE) than in other groups (p<0.05).

FIG. 3 shows the impact of serum ACE on the relation between HbA1c and the risk of severe hypoglycaemia demonstrating pronounced differences between the serum ACE quartiles throughout the clinically relevant range of HbA1c.

Mild Hypoglycaemia

No significant relation was observed between genotype (RR compared to II genotype: ID 0.8 (0.6-1.1), p=0.26 and DD 0.9 (0.6-1.3), p=0.55) or serum ACE (RR per ten unit increment of serum ACE: 0.990 (0.913-1.072), p=0.78) and frequency of mild hypoglycaemia.

References

1. Frier B M, Fisher B M, editors. Hypoglycaemia in clinical diabetes. Chichester: John Wiley & Sons; 1999.

2. Cryer P E. Hypoglycaemia is the limiting factor in the management of diabetes. Diabetes Metab Res Rev 1999;15:42-6.

3. Diabetes Control and Complications Trial Research Group. The effect of intensive treatment of diabetes on the development and progression of long-term complications in insulin-dependent diabetes mellitus. N Engl J Med 1993;329:977-86.

4. Reichard P, Pihl M. Mortality and treatment side-effects during long-term intensified conventional insulin treatment in the Stockholm Diabetes Intervention Study. Diabetes 1994;43:313-7.

5. Gold A E, MacLeod K M, Frier B M. Frequency of severe hypoglycaemia in patients with type 1 diabetes with impaired awareness of hypoglycaemia. Diabetes Care 1994;17:697-703.

6. Montgomery H E, Marshall R, Hemingway H, Myerson S, Clarkson P, Dollery C et al. Human gene for physical performance. Nature 1998;393:221-2.

7. Rigat B, Hubert C, Alhenc-Gelas F, Cambien F, Corvol P, Soubrier F. An insertion/deletion in the angiotensin I-converting enzyme gene accounting for half the variance of serum enzyme levels. J Clin Invest 1990;86:1343-6.

8. Agerholm-Larsen B, Tybjærg-Hansen A, Schnohr P, Nordestgaard B G. ACE gene polymorphism explains 30-40% of variability in serum ACE activity in both women and men in the population at large: the Copenhagen City Heart Study. Atherosclerosis 1999;147:425-7.

9. Jaspard E, Wei L, Alhenc-Gelas F. Differences in the properties and enzymatic specificities of the two acitve sites of angiotensin I-converting enzyme (kininase II). Studies with bradykinin and other natural peptides. J Biol Chem 1993;268:9496-503.

10. Gayagay G, Yu B, Hambly B, Boston T, Hahn A, Celermajer D S et al. Elite endurance athletes and the ACE I allele—the role of genes in athletic performance. Hum Genet 1998;103:48-50.

11. Myerson S, Hemingway H, Budget R, Martin J, Humphries S, Montgomery H. Human angiotensin I-converting enzyme gene and endurance performance. J Appl Physiol 1999;87:1313-6.

12. Pramming S, Pedersen-Bjergaard U, Heller S R, Wallace T, Rasmussen A K, Jørgensen H V et al. Severe hypoglycaemia in unselected patients with type 1 diabetes: a cross-sectional multicentre survey. Diabetologia 2000;43 Suppl 1:A194.

13. Pramming S, Thorsteinsson B, Bendtson I, Binder C. Symptomatic hypoglycaemia in 411 type 1 diabetic patients. Diabetic Med 1991;8:217-22.

14. The Diabetes Control and Complication Trial Research Group. Hypoglycaemia in the Diabetes Control and Complication Trial. Diabetes 1997;46:271-86.

15. Hepburn D A, Patrick A W, Eadington D W, Ewing D J, Frier B M. Unawareness of hypoglycaemia in insulin-treated diabetic patients: Prevalence and relationship to autonomic neuropathy. Diabetic Med 1990;7:711-7.

16. Clarke W L, Gonder-Frederick L, Julian D, Schlundt D, Polonsky W. Reduced awareness of hypoglycaemia in IDDM adults: a prospective study of hypoglycaemic frequency and associated symptoms. Diabetes Care 1995;18:517-22.

17. Agerholm-Larsen B, Nordestgaard B G, Steffensen R, Sørensen T I, Jensen G, Tybjærg-Hansen A. ACE gene polymorphism: ischaemic heart disease and longevity in 10, 150 individuals. A case-referent and retrospective cohort study based on the Copenhagen City Heart Study. Circulation 1997;95:2358-2367.

18. Hougaard P. Analysis of Multivariate Survival Data. Springer Verlag; 2000.

1. A method of treatment of diabetes mellitus in an individual in need thereof comprising administering to said individual a pharmaceutically effective amount of
a) at least one inhibitor of the renin-angiotensin II system; and
b) at least one antidiabetic.

2. The method according to claim 1, wherein said inhibitor of the renin-angiotensin II system is an inhibitor of angiotensin-converting enzyme (ACE).

3. The method according to claim 1, wherein said inhibitor of the renin-angiotensin II system is an inhibitor of ACE selected from the group consisting of quinapril, lisinopril, enalapril, captopril, benazepril, perindopril, trandolapril, fosinopril, meoxipril, ramipril and teprotide.

4. The method according to claim 1, wherein said inhibitor of the renin-angiotensin II system is an angiotensin II-receptor antagonist.

5. The method according to claim 1, wherein said inhibitor of the renin-angiotensin II system is an angiotensin II-receptor antagonist selected from the group consisting of candesartancilexetil, eprosartan, irbesartan, losartan, valsartan, telmisartan and saralasin.

6. The method according to claim 1, wherein said antidiabetic is human insulin or an analogue thereof or a derivative thereof.

7. The method according to claim 6, wherein said analogue of insulin is a rapid-acting analogue.

8. The method according to claim 6, wherein said analogue of insulin is selected from the group consisting of AspB28 human insulin and LysB28ProB29 human insulin.

9. The method according to claim 6, wherein the derivative is human insulin or an analogue thereof containing a C₆ to C₄₀ lipophilic substituent in position B29.

10. The method according to claim 6, wherein the derivative is selected from the group consisting of B29-N^{ε}-myristoyl-des(B30) human insulin, B29-N^{ε}-palmitoyl-des(B30) human insulin, B29-N^{ε}-myristoyl human insulin, B29-N^{ε}-palmitoyl human insulin, B28-N^{ε}-myristoyl Lys^{B28}Pro^{B29} human insulin, B28-N^{ε}-palmitoyl Lys^{B28}Pro^{B29} human insulin, B30-N^{ε}-myristoyl-Thr^{B29}Lys^{B30} human insulin, B30-N^{ε}-palmitoyl-Thr^{B29}Lys^{B30} human insulin, B29-N^{ε}-(N-palmitoyl-γ-glutamyl)-des(B30) human insulin, B29-N^{ε}-(N-lithocholyl-γ-glutamyl)-des(B30) human insulin, B29-N^{ε}-(ω-carboxyheptadecanoyl)-des(B30) human insulin and B29-N^{ε}-(ω-carboxyheptadecanoyl) human insulin.

11. The method according to claim 1, wherein said antidiabetic is an orally active hypoglycaemic agent.

12. The method according to claim 11, wherein said orally active hypoglycaemic agent is selected from the group consisting of sulfonylureas, biguanides, meglitinides, oxadiazolidinediones, thiazolidinediones, α-glucosidase inhibitors, glucagon antagonists, GLP-1 agonists, potassium channel openers, insulin sensitizers, DPP-IV inhibitors, PTPase inhibitors, inhibitors of hepatic enzymes involved in stimulation of gluconeogenesis and/or glycogenolysis, glucose uptake modulators, compounds modifying the lipid metabolism such as anti-hyperlipidemic agents and antilipidemic agents, compounds lowering food intake, PPAR and RXR agonists and agents acting on the ATP-dependent potassium channel of the β-cells.

13. The method according to claim 1, wherein said antidiabetic is selected from the group consisting of tolbutamid, pioglitazone, rosiglitazone, glibenclamid, gliclazide, glipizide, acarbose, metformin, nateglinid and repaglinid.

14. A method of preventing hypoglycaemia in an individual in need thereof comprising administering to said individual a pharmaceutical effective amount of an inhibitor of the renin-angiotensin II system.

15. The method according to claim 14, wherein the individual is suffering from diabetes mellitus.

16. The method according to claim 14, wherein said treatment is prophylactic treatment.

17. The method according to claim 14, wherein said treatment is curative treatment.

18. The method according to claim 14, wherein said treatment is ameliorating treatment.

19. The method according to claim 14, wherein the diabetes mellitus is diabetes mellitus type 1.

20. The method according to claim 14, wherein the administration is by subcutaneous injection.

21. The method according to claim 14, wherein the administration is oral administration.

22. The method according to claim 14, wherein said inhibitor of the renin-angiotensin II system is an inhibitor of angiotensin-converting enzyme (ACE).

23. The method according to claim 14, wherein said inhibitor of the renin-angiotensin II system is an inhibitor of ACE selected from the group consisting of quinapril, lisinopril, enalapril, captopril, benazepril, perindopril, trandolapril, fosinopril, meoxipril, ramipril and teprotide.

24. The method according to claim 14, wherein said inhibitor of the renin-angiotensin II system is an angiotensin II-receptor antagonist.

25. The method according to claim 14, wherein said inhibitor of the renin-angiotensin II system is an angiotensin II-receptor antagonist selected from the group consisting of candesartancilexetil, eprosartan, irbesartan, losartan, valsartan, telmisartan and saralasin.

26. A method to diagnose the susceptibility to hypoglycaemia of an individual comprising the steps of
a) obtaining a tissue sample from the individual; and
b) detecting within said tissue sample the genotype of the angiotensin-converting enzyme (ACE) gene; and
c) correlating the genotype with the susceptibility to hypoglycaemia,
or the steps of
d) obtaining a tissue sample from the individual; and
e) detecting within said tissue sample the activity of ACE; and
f) correlating said activity to the susceptibility of hypoglycaemia.

27. The method according to claim 26, wherein said tissue sample is a blood sample.

28. The method according to claim 27, wherein said blood sample is further purified to obtain serum.

29. The method according to claim 26, wherein said individual is a human being.

30. The method according to claim 26, wherein said individual is a human being suffering from diabetes mellitus.

31. The method according to claim 26, wherein said individual is a human being suffering from diabetes mellitus type 1.

32. The method according to claim 26, wherein the ACE genotype is selected from the group consisting of II, ID or DD, wherein I designates the insertion allele and D designates the deletion allele of the ACE gene.

33. The method according to claim 32, wherein the II genotype correlates with low susceptibility, the ID genotype correlates with medium susceptibility and the DD genotype correlates with high susceptibility to hypoglycaemia.

34. The method according to claim 26, wherein step b) comprises the steps of
a) purifying genomic DNA from said tissue sample; and
b) amplifying said DNA by polymerase chain reaction (PCR) using at least one pair of primers consisting of a first primer and a second primer, wherein said first primer and said second primer are specific for the ACE gene; and
c) analysing the PCR products so that the ACE genotype can be determined.

35. The method according to claim 34, wherein said first primer comprises a nucleotide sequence derived from the ACE gene sequence.

36. The method according to claim 34, wherein said first primer comprises a nucleotide sequence derived from intron 16 of the ACE gene sequence (SEQ. ID. NO: 1)

37. The method according to claim 34, wherein said second primer comprises a nucleotide sequence capable of hybridising under stringent conditions to a sequence derived from the ACE gene sequence

38. The method according to claim 34, wherein said second primer comprises a nucleotide sequence capable of hybridising under stringent conditions to a sequence derived from intron 16 of the ACE gene sequence (SEQ ID NO: 1)

39. The method according to claim 34, wherein said first primer consists of 10 to 15 nucleotides.

40. The method according to claim 34, wherein said second primer consists of 10 to 15 nucleotides.

41. The method according to claim 34, wherein said first primer comprises the nucleotide sequence 5′ CTG GAG ACC ACT CCC ATC CTT TCT 3′.

42. The method according to claim 34, wherein said second primer comprises the nucleotide sequence 5′ GAT GTG GCC ATC ACA TTC GTC AGA T 3′.

43. The method according to claim 34, wherein at least two pairs of primers are used.

44. The method according to claim 43, wherein the first primer of the second pair of primers comprises a nucleotide sequence of the ACE insertion (SEQ ID NO: 2).

45. The method according to claim 43, wherein the second primer of the second pair of primers comprises a nucleotide sequence capable of hybridising under stringent conditions to a sequence derived from the nucleotide sequence of the ACE insertion (SEQ ID NO: 2).

46. The method according to claim 26, wherein the activity of ACE is determined from a serum sample using a kinetic assay.

47. The method according to claim 46, wherein the kinetic assay is a spectrophotometric assay utilising FAPGG as substrate.

48. The method according to claim 26, wherein high ACE activity is correlated with high susceptibility and low ACE activity is correlated with low susceptibility to hypoglycaemia.

49. The method according to claim 26, wherein the risk of hypoglycaemia is increased 1.1 to 2.0 times per 10 U/l increment in serum ACE activity.

50. The method according to claim 26, wherein one or more parameters selected from the group consisting of C-peptide concentration and haemoglobin A concentration furthermore are determined.

51. A method of treatment of diabetes mellitus in an individual in need thereof comprising the steps of
a) diagnosing the susceptibility of said individual to hypoglycaemia by
i) obtaining a tissue sample from the individual; and
ii) detecting within said tissue sample the genotype of the angiotensin-converting enzyme (ACE) gene; and
iii) correlating the genotype with the susceptibility to hypoglycaemia,
or the steps of
iv) obtaining a tissue sample from the individual; and
v) detecting within said tissue sample the activity of ACE; and
vi) correlating said activity to the susceptibility of hypoglycaemia; and
b) determining an appropriate dose of an antidiabetic and/or an inhibitor of the renin-angiotensin II system to treat said individual according to said diagnosis; and
c) administering to said individual said appropriate dose of an antidiabetic.

52. The method according to claim 51, wherein said antidiabetic is human insulin or an analogue thereof or a derivative thereof.

53. The method according to claim 51, wherein said analogue of insulin is a rapid-acting analogue.

54. The method according to claim 51, wherein said analogue of insulin is selected from the group consisting of AspB28 human insulin and LysB28ProB29 human insulin.

55. The method according to claim 51, wherein the derivative is human insulin or an analogue thereof containing a C₆ to C₄₀ lipophilic substituent in position B29.

56. The method according to claim 51, wherein the derivative is selected from the group consisting of B29-N^{ε}-myristoyl-des(B30) human insulin, B29-N^{ε}-palmitoyl-des(B30) human insulin, B29-N^{ε}-myristoyl human insulin, B29-N^{ε}-palmitoyl human insulin, B28-N^{ε}-myristoyl Lys^{B28}Pro^{B29} human insulin, B28-N^{ε}-palmitoyl Lys^{B28}Pro^{B29} human insulin, B30-N^{ε}-myristoyl-Thr^{B29}Lys^{B30} human insulin, B30-N^{ε}-palmitoyl-Thr^{B29}Lys^{B30} human insulin, B29-N^{ε}-(N-palmitoyl-γ-glutamyl)-des(B30) human insulin, B29-N^{ε}-(N-lithocholyl-γ-glutamyl)-des(B30) human insulin, B29-N^{ε}-(ω-carboxyheptadecanoyl)-des(B30) human insulin and B29-N^{ε}-(ω-carboxyheptadecanoyl) human insulin.

57. The method according to claim 51, wherein said antidiabetic is an orally active hypoglycaemic agent.

58. The method according to claim 57, wherein said orally active hypoglycaemic agent is selected from the group consisting of sulfonylureas, biguanides, meglitinides, oxadiazolidinediones, thiazolidinediones, α-glucosidase inhibitors, glucagon antagonists, GLP-1 agonists, potassium channel openers, insulin sensitizers, DPP-IV inhibitors, PTPase inhibitors, inhibitors of hepatic enzymes involved in stimulation of gluconeogenesis and/or glycogenolysis, glucose uptake modulators, compounds modifying the lipid metabolism such as antihyperlipidemic agents and antilipidemic agents, compounds lowering food intake, PPAR and RXR agonists and agents acting on the ATP-dependent potassium channel of the β-cells.

59. The method according to claim 51, wherein said antidiabetic is selected from the group consisting of tolbutamid, pioglitazone, rosiglitazone, glibenclamid, gliclazide, glipizide, acarbose, metformin, nateglinid and repaglinid.

60. The method according to claim 51, wherein said administration is by subcutaneous injection.


SEQUENCE LISTING (2 sequences)

SEQ ID NO: 1 - DNA, 1856 bp, Homo sapiens
gtgagagctcatgtgcaggctgagtgagaggcgagggctgggactggcatggggcccgggggtgctgggtgagagcacagagttgggctcccctcgctcttggggtcagcgtgcccaggaaatgccctttcttgttttccacgaggggggcttctctgcccactgagagccggcacctacttcataccatgccccgatcagctgcccctccctcagaaccgccctctgcttaagggtgtccactctctcctgtcctctctgcatgccgcccctcagagcagcgggatctcaaagttatatttcatgggcttggactccaaatggggggaactcggggacactagctccccccggcctcctttcgtgaccctgcccttgacttcctcaccttctctgtctttcctgagcccctctcccagcatgtgactgataaggaaattgagtcacacagcccctgaaagcgccagactagaacctgagcctctgattcctctcacttccctcccctaccctgccacttcctactggatagaagtagacagctcttgactgtcctcttttctccccactggctggtccttcttagccccagcccgtttgaaagagctcacccccgacacaaggacccgcacacagatacctcccagctccctctcaacccaccctttccagggttggagaacttgaggcataaacattcttccatgaggaatctccacccagaaatgggtctttctggcccccagcccagctcccacattagaacaatgacaaatagaaggggaaatggaaaataaacaggagaaacggttttcccaggacagggtttggcctacaagttgtggatgtgggtacccatgccaagtgtgaggggaggctggccgggtgtggtggctcatgctctaatcccagcactttgggaggccaaggtgagtagatcacttgaggccgggagtttgagaccagcctggccaacatggtgaaaccccatctgtactaaaaatacaaaagttagctgggcgtggtggtagatgcctgtagtcccagctacttgggaggctgaggcatgagaatcgcttgagcccagccagggcaatacagcaagaccccgtctctacaaataaaatacaaaaaattagttggatgtggtggtgcatgcctgtagtcctagctgctagggaggctgagatggaaggattgcttgagcctgggaggtcaaggctgcagtgagccgagatggcgccactgcactccagcctgggcaacagagtgagaccctgtctcagaaagaaaaaaaaaaaaaaaggagaggagagagactcaagcacgcccctcacaggactgctgaggccctgcaggtgtctgcagcatgtgcccaggccggggactctgtaagccactgctggagaccactcccatcctttctcccatttctctagacctgctgcctatacagtcactttttttttttttttgagacggagtctcgctctgtcgcccaggctggagtgcagtggcgggatctcggctcactgcaacgtccgcctcccgggttcacgccattctcctgcctcagcctcccaagtagctgggaccacagcgcccgccactacgcccggctaattttttgtatttttagtagagacggggtttcaccgttttagccgggatggtctcgatctcctgacctcgtgatccgcccgcctcggcctcccaaagtgctgggattacaggcgtgatacagtcacttttatgtggtttcgccaattttattccagctctgaaattctctgagctccccttacaagcagaggtgagctaagggctggagctcaagccattcaaccccctaccag

SEQ ID NO: 2 - DNA, 288 bp, Homo sapiens
atacagtcactttttttttttttttgagacggagtctcgctctgtcgcccaggctggagtgcagtggcgggatctcggctcactgcaacgtccgcctcccgggttcacgccattctcctgcctcagcctcccaagtagctgggaccacagcgcccgccactacgcccggctaattttttgtatttttagtagagacggggtttcaccgttttagccgggatggtctcgatctcctgacctcgtgatccgcccgcctcggcctcccaaagtgctgggattacaggcgtg